(19) United States
(12) Patent Application Publication
(10) Pub. No.: US 2023/0310533 A1
(43) Pub. Date: Oct. 5, 2023

(54) ACTIVE PHARMACEUTICAL INGREDIENT

(71) Applicant: DAMIT HOLDINGS (PTY) LTD, White River (ZA)

(72) Inventor: Bajabulile Iris CONOCHIE, White River (ZA)

(21) Appl. No.: 18/207,448

(22) Filed: Jun. 8, 2023

Related U.S. Application Data
(63) Continuation of application No. PCT/ZA2021/050036, filed on May 31, 2021.

(30) Foreign Application Priority Data
Dec. 8, 2020 (ZA) 2020/07630

(51) Int. Cl. A61K 36/47; A61P 31/14

(52) U.S. Cl. CPC A61K 36/47; A61P 31/14

(57) ABSTRACT

An active pharmaceutical ingredient (API) which is prepared by a process, wherein the process comprises heating a mixture of pharmaceutical grade castor oil and sodium hydroxide, and wherein the API shows virucidal and/or fungicidal activity, in particular, to beta-corona viruses such as SAR-Co V-2.

### CROSS REFERENCE TO RELATED APPLICATIONS

This application is a continuation of International Application No.: PCT/ZA2021/050036, filed on May 31, 2021, which claims priority from South African Patent Application No. 2020/07630, filed on Dec. 8, 2020, the contents of each of which are incorporated herewith by reference.

### FIELD OF THE INVENTION

This invention relates to an active pharmaceutical ingredient (API). More particularly this invention relates to an API which has antiviral properties and, in particular, this invention relates to an antiviral API that is at least partially capable of inhibiting the proliferation of Corona viruses which are the causative agents of various types of infections, including but not limited to respiratory infections.

The API of this invention is also fungicidally active against fungal infections that may be comorbid with Corona virus infections, in particular COVID-19 infections. A person skilled in the art would appreciate the interplay between such viral infections and opportunistic fungal infections that are comorbid. Whilst such fungal infections are not often in and of themselves deadly, they can tend to weaken the immune system of a patient infected with COVID-19 to such an extent that the immune system of such patient is unable to mount a response to the viral infection thus resulting in acute respiratory distress and ultimately death.

### BACKGROUND TO THE INVENTION

Granted South Africa Patent Application ZA2016/05436 makes broad reference to the general field of application of the invention. This invention comprises a base oil that is extracted from castor oil. The base oil is then used in a number of applications for the treatment of various conditions in humans.

Castor oil comes from the castor oil plant Ricinus communis L. (Euphorbiaceae). The main component of castor oil is (R)—Ricinoleic acid (RA) which has been the focus of various studies in recent years due to its therapeutic potential (Pabis et al., 2016). The derivatives of RA have been shown to be antimicrobial and anti-tumor agents as is seen in the mentioned patent the contents of which are incorporated herein by reference.

Conoche Fatty Acids Molecules Hybridization Technology (Conoche FAM-H Technology) is based on the atomic orbital's hybridization theory, which is the concept of mixing atomic orbitals into new hybrid orbitals at a particular temperature.

Oils s are made up of three fatty acids and a glycerol. Glycerol is a backbone of fatty acids, during lipolysis fats breakdown into fatty acids and glycerol. At a particular temperature the fatty acids start to form hybrid molecules resulting in the formation of a new chemical compound described in Granted South Africa Patent Application ZA2016/05436.

Viruses that infect humans, animals, plants and even bacteria are well-known and ubiquitous. A viral disease occurs when a pathogenic virus infects susceptible cells in a human or animal body. In order to infect a cell the infectious virus particle (virion) must first adsorb to the cell surface and adsorption occurs through the interaction between viral surface proteins and receptors on the surface of susceptible cells. Once the virions are adsorbed, they penetrate the cell and they uncoat, thereby allowing the viral genetic material access to the cell machinery for replication of the virus. Once the viral components have been produced by the cell machinery, the viral particles assemble and are subsequently released from the cell, where after subsequent cells are accessed in the same manner.

Furthermore, zoonotic diseases are also well-known and it is well-known that both Ebola and HIV are the result of a zoonosis. A zoonosis event occurs when an animal virus crosses the species barrier and becomes able to infect humans.

Recently, viruses that have been able to cross the species barrier have been brought to the public's attention due to the worldwide pandemic of the corona virus disease (COVID-19). In late 2019, several individuals were hospitalized in Wuhan in China with what appeared to be cases of severe respiratory illnesses (Kannan et al., 2020). The causative agent, which is known as Severe Acute Respiratory Syndrome Corona Virus 2 (SARS-CoV-2), is a Corona virus which is previously unknown in humans, with a suspected animal source in bats and possibly pangolins (Zhu et al., 2020). The viral incubation time for SARS-CoV-2 in an infected individual is between 2 and 14 days. This is the virus that causes the disease that is now known as COVID-19, which has resulted in the worldwide pandemic causing hundreds of millions of infections and millions of deaths and severely impacting the world's economies due to the application of national lockdowns in an attempt to slow the spread of the virus by reducing the reproduction rate of the same (the so-called R-naught). Roughly, 80% of infected people show mild disease once infected, with 20% being more severely infected and a presumptive mortality rate of 5% across the globe.

SARS-CoV-2 and MERS-CoV (Middle Eastern Respiratory Syndrome Corona Virus) are members of the Coronaviridae family, this viral family being comprised of single positive stranded RNA viruses. More specifically the abovementioned viruses are part of the beta-corona virus genus (Zhu et al., 2020). This genus also includes SARS-CoV which is the causative agent of the Severe Acute Respiratory Syndrome that emerged in 2002 and spread globally in 2003 causing less than 1000 deaths and less than 10 000 known infections. Albeit that there are other viruses in the beta-corona virus genus the three mentioned are of significant importance in relation to their ability to infect humans and are closely phylogenetically related. Given that this invention has been tested specifically against SARS-CoV-2 and MERS-CoV further background on each of these viruses is given hereunder:

### SARS-CoV-2

SARS-CoV-2 is highly infectious and also easily passed between people via respiratory droplets that are produced when an infected person coughs or sneezes (Kannan et al., 2020). Notably, it has been shown that even asymptomatic infected people are able to effectively spread SAR-CoV-2 making it even more difficult to effectively limit the spread of the disease and hence amplifying the need for effective social distancing measures and sanitary procedures to be put in place across the global. Albeit that there are over 150 vaccines currently in various stage of development, and even though there are now some vaccines commercially available, serous production capacity constraints and hording of vaccines by richer countries and continues mutations of the SARS-CoV-2, implies that the first line of defense against COVID-19, at least in under developed or 3rd world countries, are still the abovementioned non-pharmaceutical interventions (in order to prevent infection), supportive therapy and the use of medicaments that are used in the treatment of other viruses and that have been shown to be effective against COVID-19. With respect to the use of known medicaments, some trials have shown that the remdesivir which is commonly used to treat Ebola has the ability to shorten a patient's recovery time from COVID-19 from 15 to 1 1 days (Grein et al., 2020) whilst others have shown that remdesivir has no effect on recovery time or mortality rate (Cohen and Kupferschmidt., 2020).

In addition to many people dying of COVID-19 higher rates of opportunistic infections have been identified in COVID-19 positive patients. These opportunistic infections are typically fungal and can include pulmonary aspergillosis, oral candidiasis or pneumocystis pneumonia (Salehi et al., 2020). Such opportunistic infections can deteriorate the status of the patients leading to acute respiratory distress and death caused by COVID-19 with a concomitantly weakened immune system. For this reason the interplay between opportunistic infections and COVID-19 should not be underestimated as exemplified in the field of the invention above.

It is therefore clear that there is a need for readily available, safe and effective pharmaceutical compositions and/or APIs for use in the treatment of SARS-CoV-2 and opportunistic fungal infections that could be associated therewith.

### MERS-CoV

MERS-CoV is a viral respiratory disease caused by a novel corona virus that emerged and was identified in Saudi Arabia in 2012, as per the records of the World Health Organisation. Also as a result of zoonosis with the presumptive animal reservoir being dromedaries. MERS-CoV is not effectively spread between people and hence the route of transmission is animal to human. To date, MERS-CoV has resulted in less than a 1000 deaths worldwide, but does show a high mortality rate of 34%. This is compared to the 9.6% mortality rate for SARS-CoV and the presumptive mortality rate for SARS-CoV-2 which is 5%, as discussed above.

Due to the close phylogenetic relationship between SARS-CoV; SARS-CoV-2 and MERS-CoV it can be hypothesized that an API that shows therapeutic efficacy against one of the aforementioned viruses may also show efficacy against the others in this beta-corona virus genus.

This specification should be interpreted within the context of the definitions set out hereunder:
Inhibitory dilution 50 (ID₅₀): The ID50 is the concentration of the test compound in pg/mL required to inhibit the virus-induced cytopathogenic effect by 50%;
Toxicity Concentration 50 (TC50): the particle concentration of the test compound that causes 50% cell mortality;
Therapeutic Index (TI): a quantitative measurement of the relative safety of a drug. It is a comparison of the amount of the test compound that causes a therapeutic effect with the amount that causes cell toxicity in test cell lines; and
The Median Tissue Culture Infectious Dose (TCID50): the concentration at which 50% of the cells are infected when a test tube or well plate upon which cells have been cultured is inoculated with a diluted solution of viral fluid. This is a means used to quantify viral titer.

### OBJECT OF THE INVENTION

It is an object of the invention to provide for an API for use in the treatment of beta-coronaviridae infections and fungal infections which are co-morbid with beta-coronaviridae infections, which at least, in part, assists in the amelioration of symptoms in infected patients.

### SUMMARY OF THE INVENTION

The active pharmaceutical ingredient (API) of the invention is prepared by a process which comprises heating a mixture of pharmaceutical grade castor oil and 1 M sodium hydroxide to a temperature of about 225° C. to about 275° C.; maintaining the mixture at a temperature of about 225° C. to about 275° C. for no more than 35 minutes to allow the castor oil and sodium hydroxide to react, cooling the reaction mixture and then centrifuging and recovering the pellet of the same, said pellet comprising the reaction product referenced hereunder.

In this instance the volumetric ratio of the castor oil to sodium hydroxide is 1:0.3 to 1:0.5.

In accordance with a first aspect of the invention there is provided an API comprising a reaction product prepared and recovered from a reaction mixture of castor oil and sodium hydroxide which is heated at a temperature of 225° C. to about 275° C. for 35 minutes, said API showing virucidal and/or fungicidal activity.

There is also provided that the API is virucidally active against Orthornavirae.

There is further provided that the API is virucidally active against Orthornavirae with positive-sense single-stranded RNA genomes.

There is also provided that the API has application in the treatment of Orthornavirae infections in humans and/or animals.

There is also provided that the positive-sense single-stranded RNA genome viruses against which the API is active include the coronaviridae and, more specifically the beta-coronaviridae.

There is also provided that the API is virucidally active against Severe Acute Respiratory Syndrome Coronavirus-2 (SARS-CoV-2).

There is further provided that the API has an ID50 of about 4347 and a Tl of about 1000 against SARS-CoV-2 in vitro.

There is also provided that the API is fungicidally active against fungal infections that are comorbid with SARS-CoV-2 infections.

There is also provided that the API is virucidally active against Middle Eastern Respiratory Syndrome Coronavirus (MERS-CoV).

There is further provided that the API has an ID50 of about 4695 and a Tl of about 1000 against MERS-CoV in vitro.

In accordance with a second aspect of the invention there is provided for the use of an API comprising a reaction product prepared from a reaction mixture of castor oil and sodium hydroxide which is heated at a temperature of 225° C. to about 275° C. for 35 minutes in the manufacture of a medicament for use in a method of treating a viral infection and/or viral infection and comorbid fungal infection.

There is also provided that for the use of the medicament in the treatment of Orthornavirae infections.

There is further provided that said treatment is applied for Orthornavirae infections in humans and/or animals.

There is further provided for the use of the medicament in the treatment of positive-sense single-stranded RNA viral infections and, in particular, betacorona virus infections.

There is further provided for the use of the medicament in the treatment of beta-corona viridae infections and particularly in the treatment of SARS-CoV-2 and MERS-CoV.

There is also provided for the use of the medicament in the treatment of fungal infections that are comorbid with SARS-CoV-2 infections.

There is further provided that said medicament is used to treat infections in humans.

In accordance with a third aspect of the invention there is provided an API for use in a method of treating viral infections and/or viral and comorbid fungal infections in humans and/or animals.

There is also provided that for the use of the API in the treatment of Orthornavirae infections.

There is further provided that said treatment is applied for Orthornavirae infections in humans and/or animals.

There is further provided for the use of the API in the treatment of positive-sense single-stranded RNA viral infections and, in particular, betacorona virus infections.

There is further provided for the use of the API in the treatment of betacorona viridae infections and particularly in the treatment of SARS-CoV-2 and MERS-CoV.

There is also provided for the use of the API in the treatment of fungal infections that are comorbid with SARS-CoV-2 infections.

There is further provided that said API is used to treat infections in humans.

In accordance with a fourth aspect of the invention there is provided a method of treating viral infections and/or comorbid fungal infections in humans and/or animals comprising administering the API to a human and/or animal in need of treatment.

There is further provided that the viral infections include Orthornavirae viral infections, more particularly positive-sense single stranded RNA viral infections and, in particular, beta-corona virus infections.

There is further provided that the beta-corona viruses comprise SAR-CoV-2 and/or MERS-CoV.

There is also provided a method of treating fungal infections that are comorbid with SARS-CoV-2 infections comprising administering the API to a human and/or animal in need thereof.

### BRIEF DESCRIPTION OF THE DRAWINGS

FIG. 1: Table showing the ID50, TC50 and Tl values of an API against SARS-CoV-2 and MERS-CoV;

FIG. 2: Graph showing the percentage inhibition of SARS-CoV-2 by the API after 72 hours. The virus was incubated with the API prior to the infection of Vero cells and after 72 hours the percentage infection inhibition was determined by measuring of luminescence. The data shown represents the results of three separate iterations with the mean and standard deviation included on the graph for reference;

FIG. 3: Graph showing the percentage inhibition of MERS-CoV by the API after 72 hours. The virus was incubated with the with the API prior to the infection of Vero cells and after 72 hours the percentage infection inhibition was determined by measuring of luminescence. The data shown represents the results of three separate iterations with the mean and standard deviation included on the graph for reference; and

FIG. 4: Graph showing the toxicity of the API against Vero cells. The cytotoxicity having been determined using the MTT assay to determine cell metabolic activity. The graph shows the results of two independent experiments together with the standard deviations and means of the same.

FIG. 5: Graph showing the body mass of Sprague-Dawley rats over the duration of the acute toxicity study using the Conoche castor oil extract as described herein.

The invention is now described by way of a number of examples set out hereunder.

### EXPERIMENTAL

### Example 1: Preparation of the Active Pharmaceutical Ingredient

As set out in ZA2016/05436 the said API (referred to as the base oil in this patent) is prepared by mixing one litre of pharmaceutical grade castor oil with 400 ml of 1 M sodium hydroxide in a closed vessel. The vessel is then placed in an unheated oven and the oven temperature is then set to a value of between 225° C.-275° C. The mixture is heated until the castor oil and the sodium hydroxide start reacting as evidenced by the boiling or frothing of the mixture. The mixture is maintained at this temperature for about 35 minutes and carefully monitored so as not to boil over.

The mixture is then removed from the oven and allowed to cool and the so-called base oil is recovered from the cooled reacted mixture which would also comprise undesired and unreacted components. The base oil is the heaviest of the reaction products and comprises the pellet of the mixture once centrifuged and therefore is easily recoverable as such.

The base oil is water soluble, is brown in colour and has a thickish oil consistency. Furthermore, this base oil has a pH>12. The further characterization of the base oil is set out in ZA2016/05436 which is incorporated herein by reference.

This base oil is the API of interest in all subsequent examples shown hereunder.

### Example 2: Virucidal Efficacy Testing

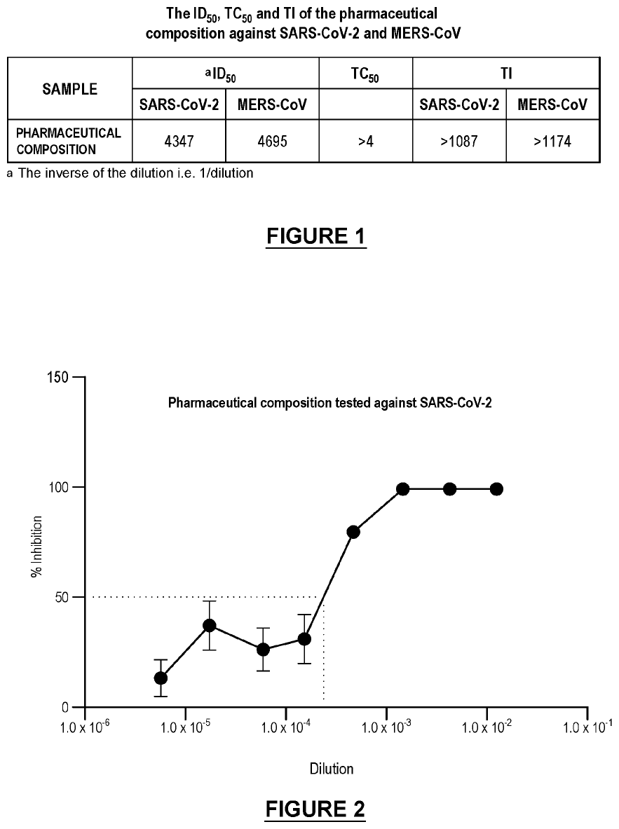

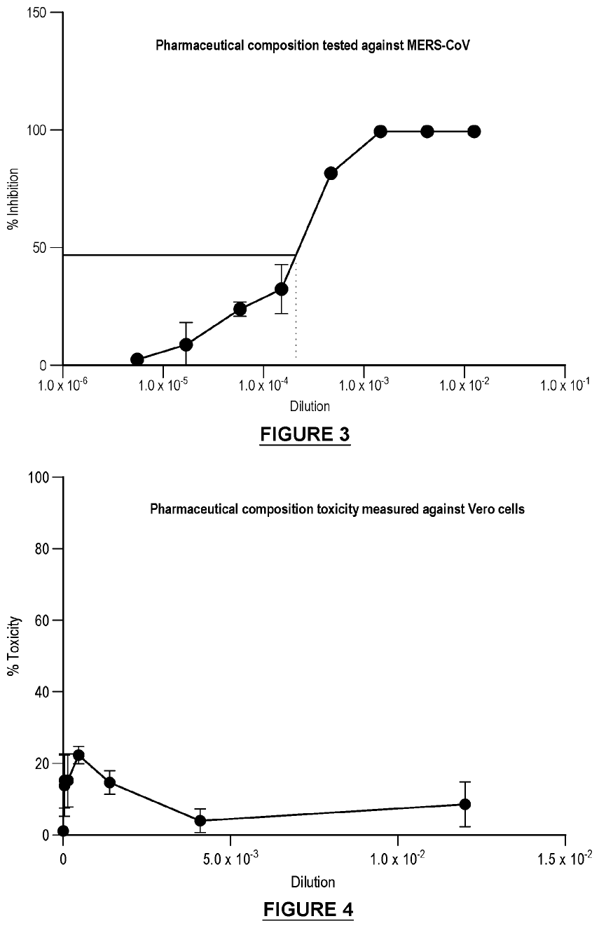

The API (namely the base oil generated in Example 1) was tested for efficacy against two coronaviruses, namely SARS-CoV-2 and MERS-CoV. The methodology of testing is set out hereunder and the results of the same are shown in FIG. 1, FIG. 2 and FIG. 3. This testing as well as the testing set out in Example 3 focused on the determination of the ID50, TC50 and the Tl of the API.

Materials
Cell lines, viral lines and media
Cell lines: Vero cells (American Type Culture Collection) and 293-T cells (American Type Culture Collection);
Cell culture growth media (Dulbecco's Modified Eagle's Medium (DMEM) supplemented with 10% Fetal bovine serum and penicillin/streptomycin);
Other cell culture components: Phosphate Buffered Saline (PBS), Trypsin-EDTA (0.25% trypsin, 1 mM EDTA), DEAE dextran;
Virus growth components:
   i. SARS-CoV-2 pseudovirus made from a plasmid encoding SARS-CoV-2 China strain envelope and a plasmid encoding the luciferase reporter gene (pNL4-3.luc.RE);
   ii. MERS-CoV was also made in the same way; only that the SARS-CoV-2 envelope was replaced with that of MERS-CoV.; Plasmid transfection reagent: FuGENE-6;
Luminescence reading reagent: Luciferase substrate (Bright Gio™ Reagent);
API to be tested;
Luminometer

Laboratory equipment and consumables
CO2 incubator;
Biosafety cabinet;
Bench top centrifuge with an adaptor for 96 well plates
Water bath
Flat bottom 96 well plates
T75 tissue culture flasks

Method

Generation of SARV-CoV-2 and MERS-CoV Pseudoviruses

SARS-CoV-2 viruses were generated in 293-T cells (2×106 cells/10 ml of growth media) by co-transfection of a construct carrying the viral envelope and a plasmid encoding the luciferase reporter gene (Wei et al., 2003). The Fugene transfection reagent was used for such transfection (Roche Applied Science, Indianapolis, IN).

The TCID50 of the virus stock was quantified by infecting Vero cells with serial 4-fold dilution of the supernatant in quadruplicate in the presence of DEAE dextran (37.5 pg/mL) (Sigma-Aldrich, St. Louis, MO).

The Bright Glo™ Reagent (Promega, Madison, WI) was used to measure infection after 72 hours of tissue culture, according to the manufacturer's instructions. Luminescence was measured in the luminometer Tecan Infinite F500.

### Assay to Investigate Oil Extract Inhibition of SARS-CoV-2 and MERS-CoV Infection of Vero Cells

A 96 well plate was used and the experiment was done in triplicate. Cell control as well as virus control wells were included. A 3-fold serial dilution was performed by adding 50 pl of the extract to 100 pl of growth media. After the dilution series, 50 pl of SARS-CoV-2 or MERS-CoV were added to all wells except the cell control wells. This was followed by incubation at 37° C. for 1 hour to allow sample to interact with the virus. Afterward 30 000 cells/100 pl/well of Vero cells were added and centrifuged at 3500 rpm for 3 hours and 30 minutes. After centrifugation, the plate was incubated at 37° C., 5% CO2 and 95% humidity for 72 hours. The incubation period was followed by the removal of substrate to all the wells. Then the plate was incubated for 2 minutes at room temperature. Afterward 150 pl was transferred to the corresponding wells of a 96-well black plate and luminescence was read using luminometer Tecan Infinite F500.

Results

The API showed in vitro activity against SARS-CoV-2 and MERS-CoV. As shown in FIG. 1 the ID50 against SARS-CoV-2 was 4347 and the ID50 against MERS-CoV was 4695.

### Example 3: Cytotoxicity Assay

The API (namely the base oil generated in Example 1) was tested for toxicity against Vero cells. The methodology of testing is set out hereunder and the results of the same are shown in FIG. 4. The materials referenced in Example 2 are applicable to this example and the methodology utilized in this example is set out hereunder:

Method

The cytotoxicity assay for the API was performed by first seeding 30 000 cells/well/100 pl of Vero cells in a 96 well plate for 24 hours at 37° C., 5% CO2 and 95% humidity. After 24 hours, a 3-fold serial dilution of the extract was performed and 100 pl was transferred to the plate containing cells, except in the cell control wells. The cells were then incubated for 72 hours. Following the 72 hours incubation the spent media was removed and replaced with fresh 180 pl growth media and 20 pl of 3-(4,5-Dimethylthiazol-2-yl)-2,5-diphenyltetrazolium bromide (MTT) reagent (5 mg/ml). This was followed by 3 hours incubation at 37° C., the removal of all media, and the addition of 150 pl of 0.1% triton X-Isopropanol. The plate was then incubated for 15 minutes at room temperature. Afterward, 100 pl of 4 mM hydrochloric acid was added and the plate was read at a wavelength of 620 nm using the Tecan Infinite F500 luminometer.

Results

The TC50 value for the API when tested against Vero cells was >4 and due to such low toxicity it was not possible to determine a precise TC50 could not be determined. The conclusion is that the API was found to be non-toxic to Vero cells.

Overall the results of the experimentation conducted in Examples 2 and 3 show that the TI for the API against both viruses was at least 1000 making the same a candidate for further testing and trialing in vivo testing.

It will be understood by a person skilled in the art that the abovementioned examples are for illustration purposes only and that the same in no way limit the scope of the invention.

### Example 4: Acute Oral Toxicity of Conoche Castor Oil Extract

### Purpose of the Study

The purpose of the study was to evaluate the acute toxicity of Conoche castor oil extract in Sprague-Dawley rats.

### Materials and Equipment

Conoche Castor Oil Extract

IVC Housing Cages
IVC blowers
Dissection kit
Round nosed oral gavage needles (16 gauge)

### Animals

Test System: Female Sprague-Dawley rats
Age at start of treatment: 9-6 weeks
Number of animals: 5
Weight: 195.49±22.30 g
Randomisation: Prior to commencement of treatment by random allocation
Identification: Earmark
Health inspection: Health inspection was performed prior to commencement of treatment

The rats were housed in enriched Individually Ventilated Cages (IVC) Rack Isolator System (equipped with input and exhaust fan filter units that provide HEPA filtered inlet and outlet air) within the PCDDP Vivarium at the Potchefstroom campus of the North-West University. Room temperature of 22±1 SC, relative humidity of 55% (±10%), a light/dark cycle of 12 hours, ventilation of 20 air changes per hour under positive pressure.

Animals were provided water ad libitum and fed standard rodent maintenance chow and housed on bedding derived from dust free and nontoxic exfoliated corncob chips in order to absorb urine, excessive moistness, and potentially hazardous ammonia vapours.

### Procedure

### Experimental Design

The study design has been adapted from the OECD 420 Acute Oral Toxicity—Fixed Dose Procedure.

### Methods

Animals were group housed and allowed to acclimatise for at least 5 days prior to administration of the test item. The animals were numbered by ear punched. Animals were weighed and then fasted overnight. A pre-study health check was performed.

The castor oil extract was administered to each animal at a dose level of 2000 mg/kg. Clinical observations determining pain and distress in laboratory rodents, were performed every 30 minutes after administration for signs of distress or discomfort during the first 4 hours, and periodically during the first 24 hours after administration. Thereafter, observations were performed daily for 14 days. The 5 animals were dosed one at a time with 24 hours in between each animal.

At the end of the study, the rats were euthanised and post mortem examination occurred.

### Data Analysis and Results

### Body Mass

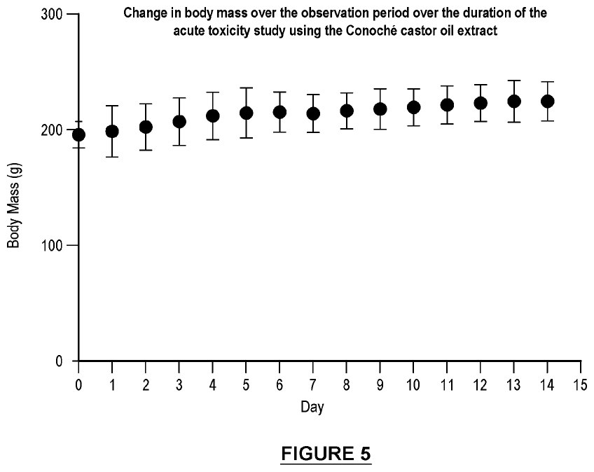

The body mass over the duration of the study is indicated in FIG. 5. Normal weight gain was observed over the observation period. Data are mean±standard deviation (n=5).

### Clinical Signs of Toxicity

No clinical signs of toxicity were observed in any of the rats exposed to the castor oil extract.

### Mortality

All the rats survived to study termination

### Necropsy Findings

No gross pathological conditions were observed and no gross lesions were found in any of the rat exposed to the castor oil extract.

### CONCLUSIONS

Rats received a single oral dose of 2000 mg/kg Conoche castor oil extract and observed over 14 days. No signs of clinical toxicity were observed and no mortality occurred over the duration of the study. Gross pathology and necropsy after euthanasia revealed no abnormalities.

### REFERENCES

1. Cohen, Y. and Kupferschmidt, K., 2020. The very, very bad look of remdesivir, the first FDA-approved COVID-19 drug. https://www.sciencemag.org/news/2020/10/very-very-bad-look-remdesivir-first-fda-approved-covid-19-drug.
2. Grein, J., Ohmagari, N., Shin, D., Diaz, G., Asperges, E., Castagna, A., Feldt, T., Green, G., Green, M. L., Lescure, F. X., Nicastri, E., Oda, R., Yo, K., Quiros-Roldan, E., Studemeister, A., Redinski, J., Ahmed, S., Bernett, J., Chelliah, D., Chen, D., Chihara, S., Cohen, S. H., Cunningham, J., D'Arminio Monforte, A., Ismail, S., Kato, H., Lapadula, G., L'Her, E., Maeno, T., Majumder, S., Massari, M., Mora-Rillo, M., Mutoh, Y, Nguyen, D., Verweij, E., Zoufaly, A., Osinusi, A. O., DeZure, A., Zhao, Y, Zhong, L., Chokkalingam, A., Elboudwarej, E., Telep, L., Timbs, L., Henne, L, Sellers, S., Cao, H., Tan, S. K., Winterbourne, L., Desai, P., Mera, R., Gaggar, A., Myers, R. P., Brainard, D. M., Childs, R., Flanigan, T., 2020. Compassionate Use of Remdesivir for Patients with Severe Covid-19. N Engl J Med.
3. Kannan, S., Shaik Syed Ali, P., Sheeza, A., Hemalatha, K., 2020. COVID-19 (Novel Coronavirus 2019)—recent trends. Ear Rev Med Pharmacol Sci 24, 2006-2011.
4. Pabis, S and Kula, J., 2016. Synthesis and bioactivity of (R)-Ricinoleic Acid derivatives: A Review. Current medicinal chemistry. 23, 4037-4056
5. Salehi, M., Almadikia, K., Badali, H., and Khodavaisy, S. 2020. Opportunistic fungal infections in the epidemic area of Covid-19: A Clinical and diagnostic perspective from Iran. Mycopathologica. July 2020.
6. Wei, X., Decker, J. M., Wang, S., Hui, H., Kappes, J. C., Wu, X., Salazar-Gonzalez, J. F., Salazar, M. G., Kilby, J. M., Saag, M. S., Komarova, N. L., Nowak, M. A., Hahn, B. H., Kwong, P. D., Shaw, G. M., 2003. Antibody neutralization and escape by HIV-1. Nature 422, 307-312.
7. Zhu, Z., Lian, X., Su, X., Wu, W., Marraro, G. A., Zeng, Y. 2020. From SARS and MERS to COVID-19: a brief summary and comparison of severe acute respiratory infections caused by three highly pathogenic human coronaviruses. Respiratory Research volume 21, Article number: 224

1. An active pharmaceutical ingredient (API), wherein the active pharmaceutical ingredient (API) is derived from the following process:
heating a mixture of pharmaceutical grade castor oil and 1 M sodium hydroxide to a temperature of about 225° C. to about 275° C.;
maintaining the mixture at a temperature of about 225° C. to about 275° C. for no more than 35 minutes to allow the castor oil and sodium hydroxide to react, and
cooling the reaction mixture and then centrifuging and recovering a pellet of the same, wherein the volumetric ratio of the castor oil to sodium hydroxide is 1:0.3 to 1:0.5, and wherein the API is virucidally active against Orthornavirae.

2. The API according to claim 1, wherein the API is virucidally active against Orthornavirae with positive-sense single-stranded RNA genomes.

4. The API according to claim 1, wherein the API has application in the treatment of Orthornavirae infections in humans and/or animals.

5. The API according to claim 2, wherein the positive-sense singlestranded RNA genome viruses against which the API is active include the coronaviridae and, more specifically the beta-coronaviridae.

6. The API according to claim 1, wherein the API is virucidally active against Severe Acute Respiratory Syndrome Coronavirus-2 (SARS-CoV-2).

7. The API according to claim 6, wherein the API has an ID50 of about 4347 and a Tl of about 1000 against SARS-CoV-2 in vitro.

8. The API according to claim 1, wherein the API is fungicidally active against fungal infections that are comorbid with SARS-CoV-2 infections.

9. The API according to claim 1, wherein the API is virucidally active against Middle Eastern Respiratory Syndrome Coronavirus (MERS-CoV).

10. The API according to claim 9, wherein the API has an ID50 of about 4695 and a Tl of about 1000 against MERS-CoV in vitro.

11. A method of use of the API according to claim 4, wherein the method is used in the manufacture of a medicament for a treatment of an Orthornavirae infection.

12. The method of use of the API according claim 4, wherein the method is used in the manufacture of a medicament for a treatment of a Orthornavirae infection and comorbid fungal infection.

13. The method of use of the API according to claim 11, wherein said treatment is applied for treating positive-sense single-stranded RNA viral infections.

14. The method of use according to claim 11, wherein the method is used for treating beta-corona virus infections.

15. The method of use according to claim 14, wherein the method is used in the treatment of SARS-CoV-2.

16. The method of use according to claim 14, wherein the method is used in the treatment of MERS-CoV.

17. The method of use according to claim 15, wherein the method is used in the treatment of fungal infections that are comorbid with SARS-CoV-2 infections.

18. The API according to claim 4, wherein the API is used in a method of treating Orthornavirae infections.

19. The API according to claim 4, wherein the API is used in a method of treating Orthornavirae infections and comorbid fungal infections.

20. The API according to claim 18, wherein the API is used in the treatment of positivesense single-stranded RNA viral infections.

21. The API according to claim 18, wherein the API is used in the treatment of betacorona virus infections.

22. The API according to claim 18, wherein the API is used in the treatment of SARS-CoV-2.

23. The API according to claim 18, wherein the API is used in the treatment of MERS-CoV.

24. The API according to claim 22, wherein the API is used in the treatment of fungal infections that are comorbid with SARS-CoV-2 infections.

25. A method of treating Orthornavirae infections in humans and/or animals comprising administering the API according to claim 1 to a human and/or animal in need thereof.

26. The method of claim 25, wherein a viral infection is a positive-sense single stranded RNA viral infections.

27. The method of claim 26, wherein the positive-sense single stranded RNA viral infection is a beta-corona virus infection.

28. The method of claim 27, wherein the beta-corona virus is SAR-CoV-2.

29. The method of claim 27, wherein the beta-corona virus is MERS-CoV.
